(19) United States
(12) Patent Application Publication
(10) Pub. No.: US 2025/0191251 A1
(43) Pub. Date: Jun. 12, 2025

(54) X-RAY CT APPARATUS AND TOMOGRAPHIC IMAGE GENERATION METHOD

(71) Applicant: FUJIFILM Corporation, Tokyo (JP)

(72) Inventors: Kazuya SADAKATA, Tokyo (JP); Taiga GOTO, Tokyo (JP)

(73) Assignee: FUJIFILM Corporation, Tokyo (JP)

(21) Appl. No.: 18/945,475

(22) Filed: Nov. 12, 2024

(30) Foreign Application Priority Data
Dec. 11, 2023 (JP) 2023-208419

(51) Int. Cl. G06T 11/00

(52) U.S. Cl. CPC G06T 11/005; G06T 2207/10081

(57) ABSTRACT

Provided are an X-ray CT apparatus and a tomographic image generation method capable of suppressing cone beam artifacts and noise without acquiring projection data in a plurality of energy bins.

An X-ray CT apparatus includes: a data acquisition unit that acquires first projection data corresponding to a first projection angle range and second projection data corresponding to a second projection angle range that is a projection angle range wider than the first projection angle range; an image correction unit that generates a first corrected image by reducing high-frequency components of a first tomographic image reconstructed from the first projection data and that generates a second corrected image by reducing low-frequency components of a second tomographic image reconstructed from the second projection data; and an image synthesis unit that synthesizes the first corrected image and the second corrected image.

### CLAIM OF PRIORITY

The present application claims priority from Japanese Patent Application JP 2023-208419 filed on Dec. 11, 2023, the content of which is hereby incorporated by reference into this application.

### BACKGROUND OF THE INVENTION

### 1. Field of the Invention

The present invention relates to a technique for handling a tomographic image obtained by an X-ray computed tomography (CT) apparatus, and particularly, to a technique for suppressing artifacts of the tomographic image.

### 2. Description of the Related Art

An X-ray CT apparatus is an apparatus that reconstructs a tomographic image using projection data acquired at a plurality of projection angles by rotating an X-ray source, which irradiates a subject with X-rays, and a detector, which detects X-rays transmitted through the subject, around the subject. The reconstructed tomographic image is used for image diagnosis as a medical image. In a cone beam CT apparatus in which X-ray irradiation is performed at a wide cone angle and the detector is multi-rowed in a body axis direction of the subject, a time for acquiring projection data can be shortened, but cone beam artifacts that hinder image diagnosis may occur.

JP2023-508147A discloses a method and a system that suppress cone beam artifacts without increasing noise in a wide cone angle spectral CT apparatus. Specifically, it is disclosed that the projection data acquired in a plurality of energy bins is decomposed in projection domains, and noise or inconsistency leading to cone beam artifacts is unevenly dispersed to a plurality of sinograms obtained through the decomposition.

### SUMMARY OF THE INVENTION

However, JP2023-508147A does not take into consideration an X-ray CT apparatus that cannot acquire projection data in a plurality of energy bins. In order to acquire the projection data in a plurality of energy bins, a dual-source CT apparatus, a photon-counting CT apparatus, or the like is required.

In that respect, an object of the present invention is to provide an X-ray CT apparatus and a tomographic image generation method capable of suppressing cone beam artifacts and noise without acquiring projection data in a plurality of energy bins.

In order to achieve the above-described object, according to an aspect of the present invention, there is provided an X-ray CT apparatus comprising: a data acquisition unit that acquires first projection data corresponding to a first projection angle range and second projection data corresponding to a second projection angle range that is a projection angle range wider than the first projection angle range; an image correction unit that generates a first corrected image by reducing high-frequency components of a first tomographic image reconstructed from the first projection data and that generates a second corrected image by reducing low-frequency components of a second tomographic image reconstructed from the second projection data; and an image synthesis unit that synthesizes the first corrected image and the second corrected image.

In addition, according to another aspect of the present invention, there is provided an X-ray CT apparatus comprising: a data acquisition unit that acquires first projection data corresponding to a first projection angle range and second projection data corresponding to a second projection angle range that is a projection angle range wider than the first projection angle range; a data correction unit that generates first corrected data by reducing high-frequency components of the first projection data and that generates second corrected data by reducing low-frequency components of the second projection data; and a reconstruction unit that reconstructs a tomographic image from synthesized data obtained by synthesizing the first corrected data and the second corrected data.

Further, according to still another aspect of the present invention, there is provided a tomographic image generation method comprising: a data acquisition step of acquiring first projection data corresponding to a first projection angle range and second projection data corresponding to a second projection angle range that is a projection angle range wider than the first projection angle range; an image correction step of generating a first corrected image by reducing high-frequency components of a first tomographic image reconstructed from the first projection data and of generating a second corrected image by reducing low-frequency components of a second tomographic image reconstructed from the second projection data; and an image synthesis step of synthesizing the first corrected image and the second corrected image.

According to the aspects of the present invention, it is possible to provide an X-ray CT apparatus and a tomographic image generation method capable of suppressing cone beam artifacts and noise without acquiring projection data in a plurality of energy bins.

### BRIEF DESCRIPTION OF THE DRAWINGS

FIG. 1 is an overall configuration diagram of an X-ray CT apparatus.

FIG. 2 is a diagram showing a configuration of projection data.

FIG. 3 is a diagram showing an example of a processing flow for Example 1.

FIG. 4 is a diagram showing an example of a processing flow for corrected image reconstruction in Example 1.

FIG. 5 is a diagram showing an example of half scan data and full scan data.

FIG. 6 is a diagram showing an example of a processing flow for corrected image reconstruction in Example 2.

FIG. 7 is a diagram showing an example of a processing flow for corrected image reconstruction in Example 3.

FIG. 8 is a diagram showing an example of first half scan data and second half scan data.

### DESCRIPTION OF THE PREFERRED EMBODIMENTS

Hereinafter, examples of an X-ray CT apparatus and a tomographic image generation method according to the present invention will be described with reference to the accompanying drawings. In the following description and accompanying drawings, components having the same functional configuration will be assigned the same reference numerals, and duplicate descriptions thereof will not be repeated.

### Example 1

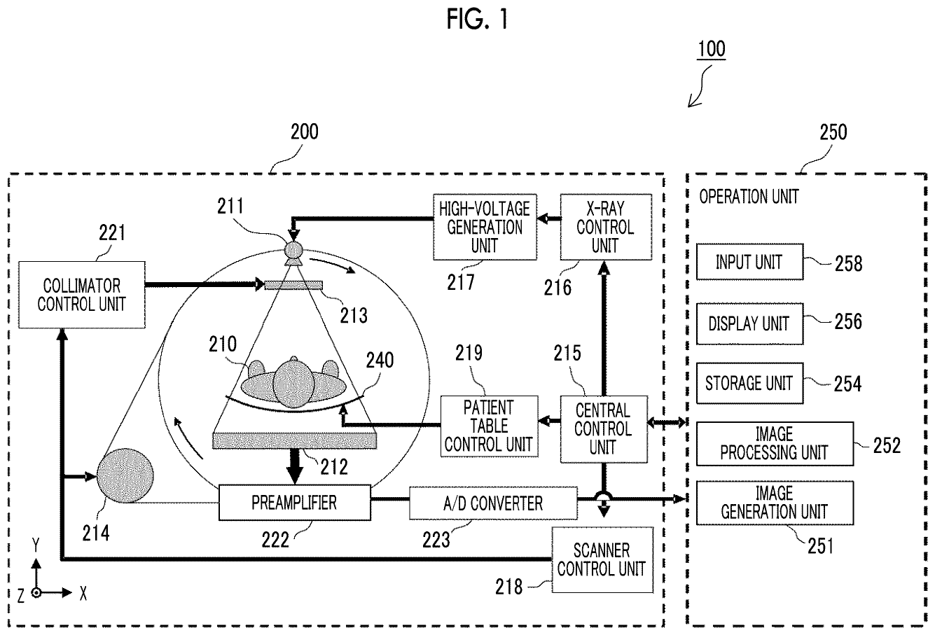

The overall configuration of an X-ray CT apparatus 100 will be described with reference to FIG. 1. In FIG. 1, a horizontal direction is denoted as an X axis, a vertical direction is denoted as a Y axis, and a direction perpendicular to a paper surface is denoted as a Z axis. The X-ray CT apparatus 100 comprises a scanner 200 and an operation unit 250. The scanner 200 includes an X-ray tube 211, a detector 212, a collimator 213, a drive unit 214, a central control unit 215, an X-ray control unit 216, a high-voltage generation unit 217, a scanner control unit 218, a patient table control unit 219, a collimator control unit 221, a preamplifier 222, an A/D converter 223, a patient table 240, and the like.

The X-ray tube 211 is a device that irradiates a subject 210 placed on the patient table 240 with X-rays. A high voltage generated by the high-voltage generation unit 217 is applied to the X-ray tube 211 in accordance with a control signal transmitted from the X-ray control unit 216, thereby irradiating the subject with X-rays from the X-ray tube 211.

The collimator 213 is a device that limits an irradiation range of the X-rays emitted from the X-ray tube 211. The irradiation range of the X-rays is set in accordance with a control signal transmitted from the collimator control unit 221.

The detector 212 is a device that measures a spatial distribution of transmitted X-rays by detecting X-rays that have been transmitted through the subject 210. The detector 212 is disposed to face the X-ray tube 211, and a large number of detection elements are two-dimensionally arranged in a plane facing the X-ray tube 211. A signal measured by the detector 212 is amplified by the preamplifier 222 and then is converted into a digital signal by the A/D converter 223. After that, various types of correction processing are performed on the digital signal, thereby acquiring projection data.

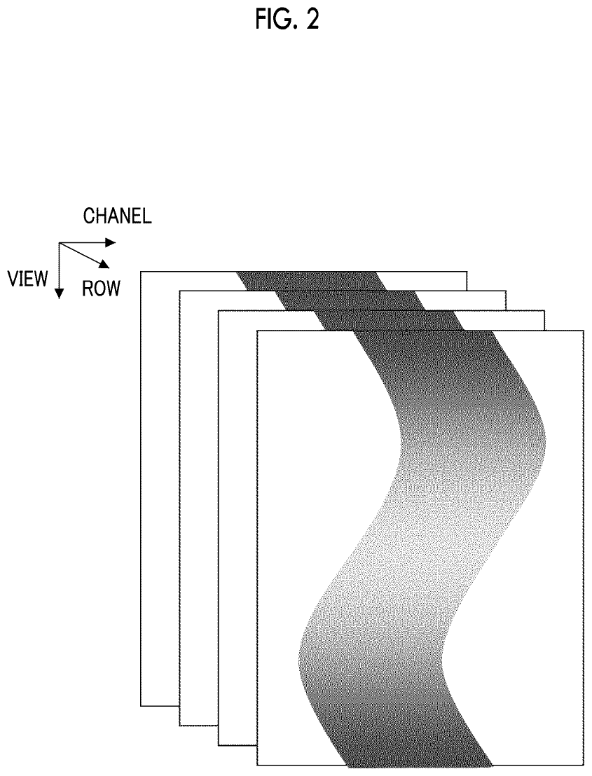

The drive unit 214 rotates the X-ray tube 211 and the detector 212 around the subject 210 in accordance with a control signal transmitted from the scanner control unit 218. The irradiation and the detection of the X-rays are performed with the rotation of the X-ray tube 211 and the detector 212, thereby acquiring projection data from a plurality of projection angles. A unit for collecting data for each projection angle is referred to as a view. In the arrangement of the two-dimensionally arranged detection elements of the detector 212, a rotation direction of the detector 212 is referred to as a channel, and a direction orthogonal to the channel is referred to as a row. As illustrated in FIG. 2, the projection data is identified by the view, the channel, and the row. In addition, a divergence angle of the X-rays emitted from the X-ray tube 211 in a channel direction is referred to as a fan angle.

The patient table control unit 219 controls an operation of the patient table 240 to keep the patient table 240 stationary or move the patient table 240 at a constant speed in a Z-axis direction, which is a body axis direction of the subject 210, while the irradiation and the detection of the X-rays are performed. A scan with the patient table 240 kept stationary is referred to as an axial scan, and a scan performed while moving the patient table 240 is referred to as a helical scan.

The central control unit 215 is a device that controls an operation of the scanner 200 described above in accordance with an instruction from the operation unit 250, and specifically, is a central processing unit (CPU), a micro processor unit (MPU), or the like.

The operation unit 250 will be described. The operation unit 250 includes an image generation unit 251, an image processing unit 252, a storage unit 254, a display unit 256, an input unit 258, and the like.

The image generation unit 251 is a device that reconstructs a tomographic image by using the projection data acquired by the scanner 200, and specifically, is a CPU, a graphics processing unit (GPU), or the like. The image processing unit 252 is a device that performs various types of image processing in order to make the tomographic image suitable for diagnosis, and specifically, is a CPU, a GPU, or the like.

The storage unit 254 is a device that stores the projection data, the tomographic image, and an image after image processing, and specifically, is a hard disk drive (HDD), a solid state drive (SSD), or the like. The display unit 256 is a device that displays the tomographic image or the image after image processing, and specifically, is a liquid crystal display or the like. The input unit 258 is a device that is used in a case in which an operator sets acquisition conditions (a tube voltage, a tube current, a scan speed, and the like) for the projection data and reconstruction conditions (a reconstruction filter, a FOV size, and the like) for the tomographic image, and specifically, is a keyboard, a mouse, a touch panel, and the like. The mouse may be another pointing device, such as a trackpad or a trackball.

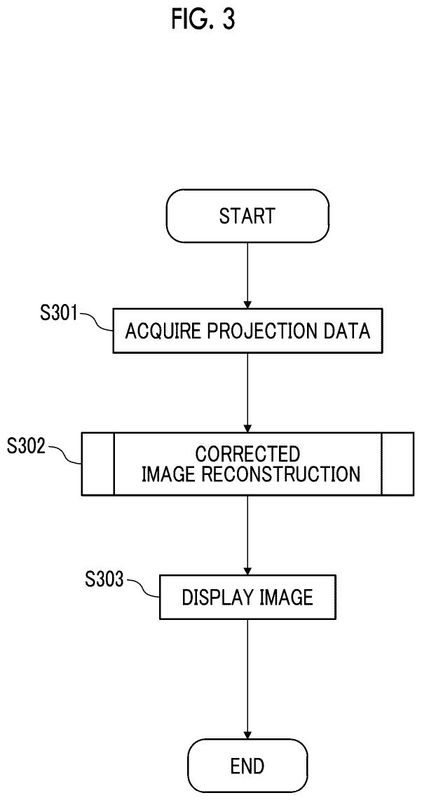

An example of a processing flow executed in Example 1 will be described step by step with reference to FIG. 3.

### S301

The image generation unit 251 acquires the projection data. The projection data may be calculated based on detector outputs output by the detector 212 or may be read out from the storage unit 254.

### S302

The image generation unit 251 reconstructs the tomographic image by using the projection data acquired in S301. The tomographic image reconstructed in S302 is corrected such that cone beam artifacts and noise are suppressed.

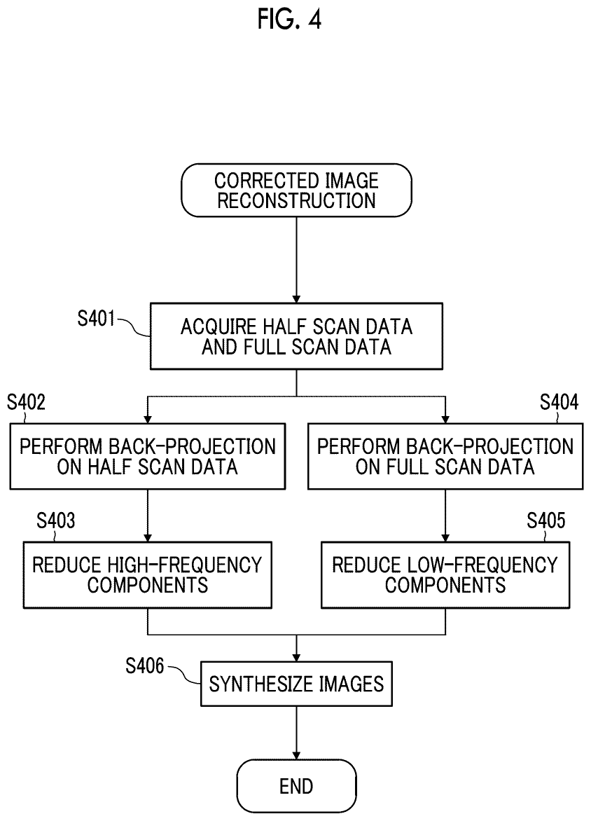

An example of a processing flow for corrected image reconstruction in S302 will be described step by step with reference to FIG. 4.

### S401

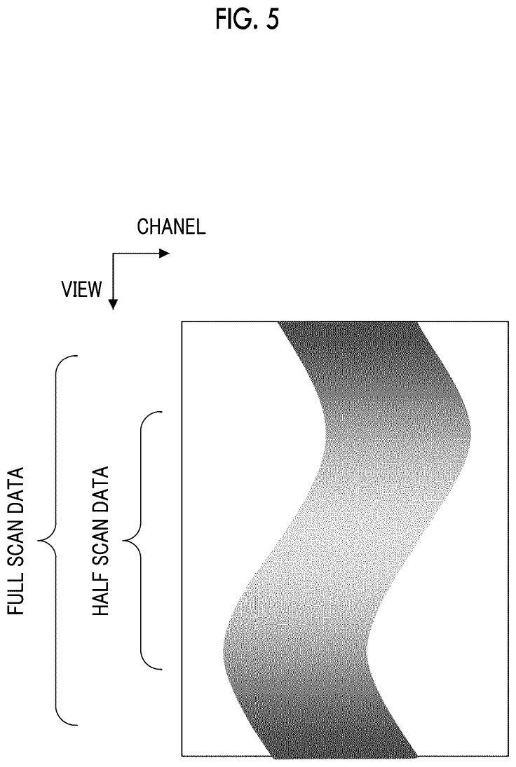

The image generation unit 251 acquires half scan data and full scan data from the projection data acquired in S301. The half scan data is projection data with a projection angle range of 180 degrees or more and less than 360 degrees, and for example, in a case in which the fan angle of the X-ray CT apparatus 100 is q, the projection data has a projection angle range of 180 degrees+φ. The full scan data is projection data with a projection angle range that is wider than that of the half scan data, and is, for example, projection data with a projection angle range that is wider than 180 degrees+φ. FIG. 5 shows an example of the half scan data and the full scan data. In the half scan data and the full scan data illustrated in FIG. 5, the centers of the views coincide with each other.

### S402

The image generation unit 251 reconstructs a half scan image as a first tomographic image by performing back-projection on the half scan data acquired in S401.

### S403

The image generation unit 251 generates a first corrected image by reducing high-frequency components of the half scan image reconstructed in S402. In order to reduce the high-frequency components, any low-pass filter, for example, a Gaussian filter is used. The high-frequency components and low-frequency components are separated from each other by, for example, a Nyquist frequency of the X-ray CT apparatus.

The half scan image has relatively fewer cone beam artifacts but has relatively higher noise. Therefore, the first corrected image generated by reducing the high-frequency components is an image with relatively fewer cone beam artifacts and suppressed noise.

### S404

The image generation unit 251 reconstructs a full scan image as a second tomographic image by performing back-projection on the full scan data acquired in S401.

### S405

The image generation unit 251 generates the second corrected image by reducing the low-frequency components of the full scan image reconstructed in S404. In order to reduce the low-frequency components, any high-pass filter is used. The second corrected image may be generated by, for example, subtracting an image obtained by applying a Gaussian filter to the full scan image from the full scan image. In a case in which the Gaussian filter is used to reduce the high-frequency components in S403, the same Gaussian filter is used to reduce the low-frequency components in S405.

The full scan image has relatively less noise but has relatively more cone beam artifacts. Therefore, the second corrected image generated by reducing the low-frequency components is an image with relatively less noise and suppressed cone beam artifacts.

### S406

The image generation unit 251 synthesizes the first corrected image generated in S403 and the second corrected image generated in S405. A synthesized image is generated by, for example, adding the first corrected image and the second corrected image. The synthesized image may be generated by performing weighted addition of the first corrected image and the second corrected image. A weight coefficient used for the weighted addition may be set in advance for each site.

The processing flow illustrated in FIG. 4 allows the first corrected image with relatively fewer cone beam artifacts and suppressed noise and the second corrected image with relatively less noise and suppressed cone beam artifacts to be synthesized. As a result, a synthesized image with suppressed cone beam artifacts and noise can be generated. Returning to the description of FIG. 3.

### S303

The image generation unit 251 displays the tomographic image generated in S302 on the display unit 256. The tomographic image displayed on the display unit 256 is used for image diagnosis.

The processing flow illustrated in FIG. 3 enables a tomographic image with suppressed cone beam artifacts and noise to be displayed on the display unit 256, allowing smooth progress in image diagnosis.

### Example 2

In Example 1, the corrected image reconstruction through the synthesis of the half scan image with reduced high-frequency components and the full scan image with reduced low-frequency components has been described. The corrected image reconstruction is not limited to the processing flow illustrated in FIG. 4. In Example 2, corrected image reconstruction through reconstruction of scan data obtained by synthesizing half scan data with reduced high-frequency components and full scan data with reduced low-frequency components will be described. Since the present example is the same as Example 1 except for the processing flow of the corrected image reconstruction, the description thereof will not be repeated.

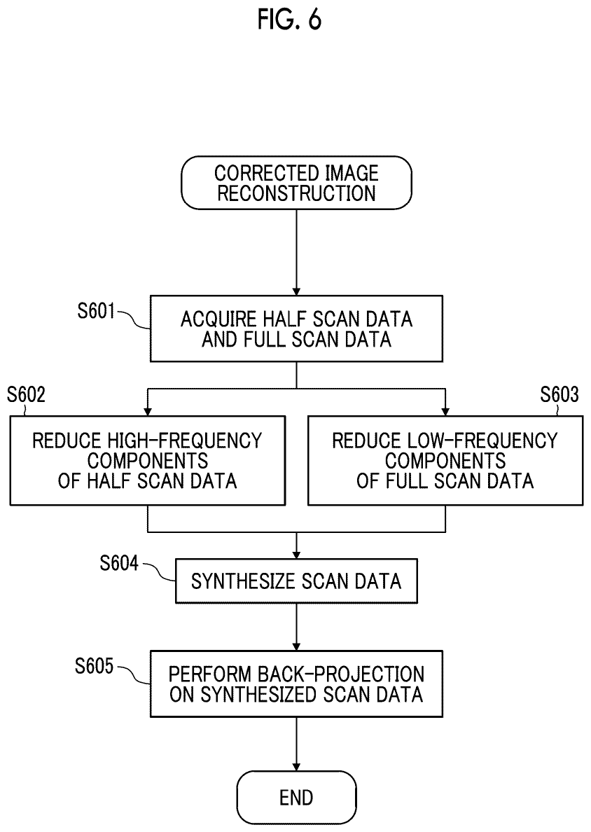

An example of a processing flow for corrected image reconstruction in Example 2 will be described step by step with reference to FIG. 6.

### S601

As in S401, the image generation unit 251 acquires the half scan data and the full scan data from the projection data acquired in S301.

### S602

The image generation unit 251 generates first corrected data by reducing high-frequency components of the half scan data acquired in S601. The reduction of the high-frequency components is performed for each view, and any low-pass filter is used.

### S603

The image generation unit 251 generates second corrected data by reducing low-frequency components of the full scan data acquired in S601. The reduction of the low-frequency components is performed for each view, and any high-pass filter is used. Note that the high-pass filter and the low-pass filter are set such that the sum of the high-pass filter used in S603 and the low-pass filter used in S602 is 1.

### S604

The image generation unit 251 synthesizes the first corrected data generated in S602 and the second corrected data generated in S603. Synthesized data is generated by, for example, adding the first corrected data and the second corrected data. The synthesized data may be generated by performing weighted addition of the first corrected data and the second corrected data. A weight coefficient used for the weighted addition may be set in advance for each site.

### S605

The image generation unit 251 reconstructs the tomographic image by performing back-projection on the synthesized data generated in S604.

The processing flow illustrated in FIG. 6 enables a tomographic image with suppressed cone beam artifacts and noise to be reconstructed. In the processing flow of FIG. 6, since the number of back-projections is one, which is fewer than two back-projections in FIG. 4, the calculation time can be shortened. However, in the processing flow of FIG. 4, the high-frequency components and the low-frequency components are reduced with respect to the tomographic image, which requires less memory for the calculation as compared to reducing the high-frequency components and the low-frequency components with respect to the projection data as shown in FIG. 6.

### Example 3

In Example 1, the corrected image reconstruction through the synthesis of the half scan image with reduced high-frequency components and the full scan image with reduced low-frequency components has been described. In Example 3, a case will be described in which a full scan pseudo-image corresponding to the full scan image is generated by using two half scan images and the full scan pseudo-image with reduced low-frequency components is used for the corrected image reconstruction. Since the present example is the same as Example 1 except for the processing flow of the corrected image reconstruction, the description thereof will not be repeated.

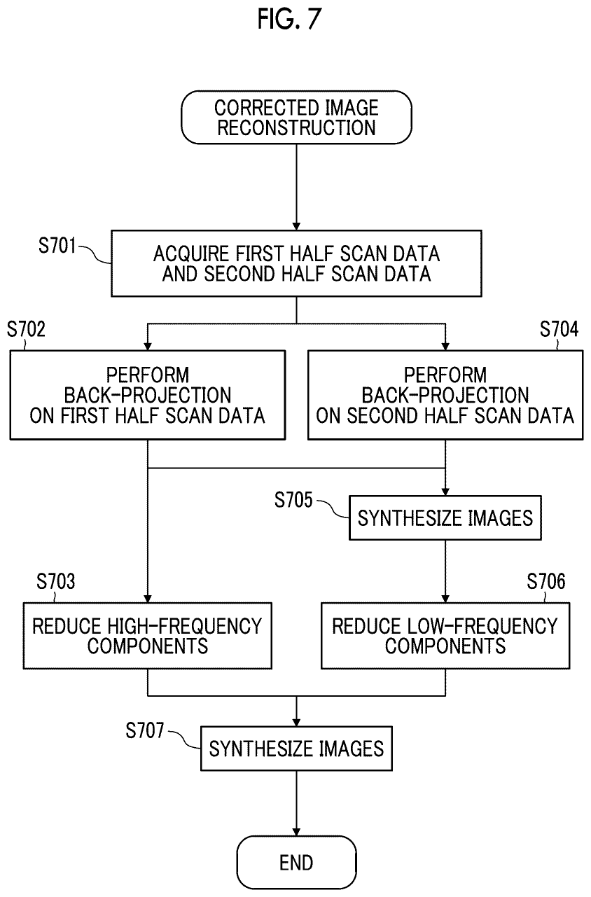

An example of a processing flow for corrected image reconstruction in Example 3 will be described step by step with reference to FIG. 7.

### S701

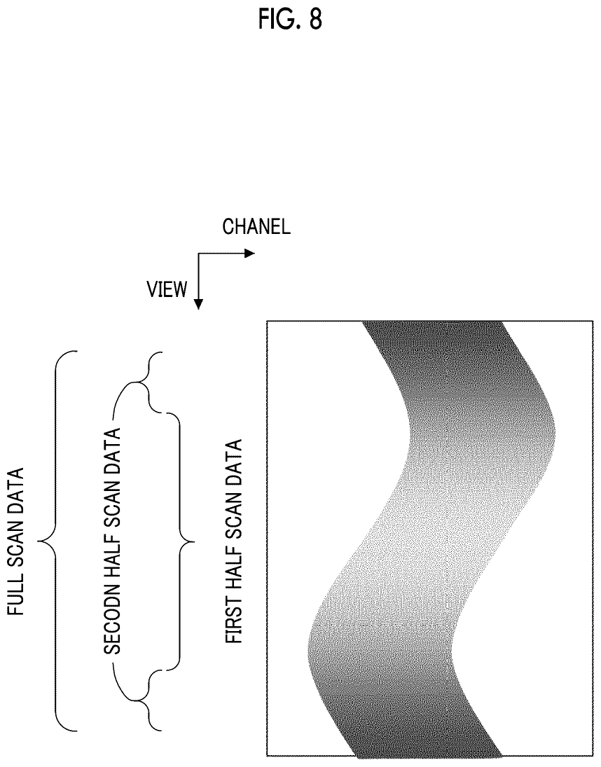

The image generation unit 251 acquires first half scan data and second half scan data from the projection data acquired in S301. An example of the first half scan data and the second half scan data will be described with reference to FIG. 8. The first half scan data is projection data with a projection angle range of 180 degrees or more and less than 360 degrees. The second half scan data is projection data in which the first half scan data is removed from the full scan data.

### S702

The image generation unit 251 reconstructs a first half scan image as the first tomographic image by performing back-projection on the first half scan data acquired in S701.

### S703

The image generation unit 251 generates a first corrected image by reducing high-frequency components of the first half scan image reconstructed in S702. In order to reduce the high-frequency components, any low-pass filter, for example, a Gaussian filter is used.

The first half scan image has relatively fewer cone beam artifacts but has relatively higher noise. Therefore, the first corrected image generated by reducing the high-frequency components is an image with relatively fewer cone beam artifacts and suppressed noise.

### S704

The image generation unit 251 reconstructs a second half scan image as a third tomographic image by performing back-projection on the second half scan data acquired in S701.

### S705

The image generation unit 251 generates the full scan pseudo-image, which is an image corresponding to the full scan image, by synthesizing the first half scan image reconstructed in S702 and the second half scan image reconstructed in S704. The full scan pseudo-image is generated by, for example, adding the first half scan image and the second half scan image.

### S706

The image generation unit 251 generates the second corrected image by reducing low-frequency components of the full scan pseudo-image generated in S705. In order to reduce the low-frequency components, any high-pass filter is used. The second corrected image may be generated by, for example, subtracting an image obtained by applying a Gaussian filter to the full scan pseudo-image from the full scan pseudo-image. In a case in which the Gaussian filter is used to reduce the high-frequency components in S703, the same Gaussian filter is used to reduce the low-frequency components in S706.

The full scan pseudo-image has relatively less noise but has relatively more cone beam artifacts. Therefore, the second corrected image generated by reducing the low-frequency components is an image with relatively less noise and suppressed cone beam artifacts.

### S707

The image generation unit 251 synthesizes the first corrected image generated in S703 and the second corrected image generated in S706. A synthesized image is generated by, for example, adding the first corrected image and the second corrected image. The synthesized image may be generated by performing weighted addition of the first corrected image and the second corrected image. A weight coefficient used for the weighted addition may be set in advance for each site.

The processing flow illustrated in FIG. 7 allows the first corrected image with relatively fewer cone beam artifacts and suppressed noise and the second corrected image with relatively less noise and suppressed cone beam artifacts to be synthesized. As a result, a synthesized image with suppressed cone beam artifacts and noise can be generated. In the processing flow of FIG. 7, although the number of back-projections is two, both the back-projections are performed on the half scan data. Therefore, the calculation time can be shortened as compared with FIG. 4 in which back-projections are performed on the half scan data and the full scan data.

The examples of the present invention have been described above. Note that the present invention is not limited to the above-described examples, and the components can be modified and embodied without departing from the gist of the invention. Additionally, a plurality of components disclosed in the above-described examples may be combined as appropriate. Furthermore, some components may be deleted from all the components described in the above-described examples.

### EXPLANATION OF REFERENCES

100: X-ray CT apparatus
200: scanner
210: subject
211: X-ray tube
212: detector
213: collimator
214: drive unit
215: central control unit
216: X-ray control unit
217: high-voltage generation unit
218: scanner control unit
219: patient table control unit
221: collimator control unit
222: preamplifier
223: A/D converter
240: patient table
250: operation unit
251: image generation unit
252: image processing unit
254: storage unit
256: display unit
258: input unit

What is claimed is:

1. An X-ray CT apparatus comprising:
a data acquisition unit that acquires first projection data corresponding to a first projection angle range and second projection data corresponding to a second projection angle range that is a projection angle range wider than the first projection angle range;
an image correction unit that generates a first corrected image by reducing high-frequency components of a first tomographic image reconstructed from the first projection data and that generates a second corrected image by reducing low-frequency components of a second tomographic image reconstructed from the second projection data; and
an image synthesis unit that synthesizes the first corrected image and the second corrected image.

2. An X-ray CT apparatus comprising:
a data acquisition unit that acquires first projection data corresponding to a first projection angle range and second projection data corresponding to a second projection angle range that is a projection angle range wider than the first projection angle range;
a data correction unit that generates first corrected data by reducing high-frequency components of the first projection data and that generates second corrected data by reducing low-frequency components of the second projection data; and
a reconstruction unit that reconstructs a tomographic image from synthesized data obtained by synthesizing the first corrected data and the second corrected data.

3. The X-ray CT apparatus according to claim 1,
wherein the data acquisition unit further acquires third projection data in which the first projection data is removed from the second projection data, and
the image correction unit generates the second corrected image by synthesizing a third tomographic image reconstructed from the third projection data and the first tomographic image.

4. A tomographic image generation method comprising:
a data acquisition step of acquiring first projection data corresponding to a first projection angle range and second projection data corresponding to a second projection angle range that is a projection angle range wider than the first projection angle range;
an image correction step of generating a first corrected image by reducing high-frequency components of a first tomographic image reconstructed from the first projection data and of generating a second corrected image by reducing low-frequency components of a second tomographic image reconstructed from the second projection data; and
an image synthesis step of synthesizing the first corrected image and the second corrected image.

5. The tomographic image generation method according to claim 4,
wherein, in the data acquisition step, third projection data in which the first projection data is removed from the second projection data is further acquired, and
in the image correction step, the second corrected image is generated by synthesizing a third tomographic image reconstructed from the third projection data and the first tomographic image.
